(19) United States
(12) Patent Application Publication
(10) Pub. No.: US 2008/0009890 A1
(43) Pub. Date: Jan. 10, 2008

(54) Scalpel with retractable blade

(76) Inventor: Robert Gerard Holman, Sandton (ZA)

Correspondence Address: MEYERTONS, HOOD, KIVLIN, KOWERT & GOETZEL, P.C., P.O. BOX 398, AUSTIN, TX, 78767-0398, US

(21) Appl. No.: 11/520,226

(22) Filed: Sep. 13, 2006

(30) Foreign Application Priority Data
Jul. 6, 2006 (ZA) 2006/05564

(51) Int. Cl. A61B 17/32

(52) U.S. Cl. 606/167

(57) ABSTRACT

A scalpel is provided having a handle with a longitudinally extending cavity therein, and a blade carrier movable longitudinally within the cavity relative to the handle between an operative position in which a blade carried thereby is in an extended position in which it is exposed for use at an open end of the cavity and a retracted inoperative position in which a blade carried thereby is retracted within the cavity in the handle. The blade carrier is movable by means of a manually operable slider attached to it by way of a slot in a wall of the handle. The slider is of generally channel shape in cross-section so as to have a web section that bridges an edge of the handle and a flange section located one on each side of the handle so that the slider is operable, by the operative engagement of a thumb or finger therewith, from the top of the scalpel handle as well as from either side thereof.

### FIELD OF THE INVENTION

This invention relates to a scalpel having a retractable blade and, more particularly, but not exclusively, to a disposable scalpel in which movement of the blade relative to a supporting scalpel handle between an extended operative position and a retracted inoperative position is achieved by moving a blade carrier by way of a thumb or finger operable slider attached, generally by way of a slot through the wall of the handle, to a blade carrier.

### BACKGROUND TO THE INVENTION

Disposable scalpels having retractable blades have been proposed and produced in many different forms. In one form the thumb operable slider projects through a sidewall of the scalpel handle, and in a second form the slider projects through a slot in one edge of the handle that can be considered to be the top edge of the scalpel handle.

In instances in which the slider projects through a sidewall of the handle the difficulty is that a scalpel designed for use by a right-handed person cannot be easily used by a left-handed person and vice versa. U.S. Pat. No. 6,254,621 describes a scalpel that is typical of this type.

In instances in which the slider projects through the top edge of the scalpel handle the thumb of a person has generally to be moved from a side of the scalpel to the top in order to operate the slider and the movement required of a persons thumb may not be convenient whilst maintaining a proper grip on the scalpel.

### OBJECT OF THE INVENTION

It is an object of this invention to provide a scalpel with a retractable blade that has a slider that is more convenient for operation and that is also equally suitable for use by both left-handed and right-handed persons.

### SUMMARY OF THE INVENTION

In accordance with this invention there is provided a scalpel having a handle with a longitudinally extending cavity therein, a blade carrier within the cavity and movable longitudinally relative to the handle between an operative position in which a blade carried thereby is exposed for use at an open end of the cavity and an inoperative position in which a blade carried thereby is retracted within the cavity in the handle, and a manually operable slider associated with the blade carrier and passing through a slot in a wall of the handle, the scalpel being characterized in that the slider is of generally channel shape in cross-section so as to have a web section and a flange section on each side thereof and wherein the web section bridges an edge of the handle (herein termed the top edge) and the flange sections are located one on each side of the top edge such that the slider is operable from the top of the scalpel handle as well as from substantially either side of the scalpel handle.

Further features of the invention provide for the slot to extend along said top edge of the handle; for the slider and one or other longitudinally extending edge of the slot to be provided with co-operating tooth and notch formations that cooperate to releasably hold the blade carrier and slider assembly in “click-stop” manner in the operative or inoperative positions, and optionally both; and for the scalpel to be generally as described in United States patent application publication number US 2004/0158269 A1, the disclosure of which is included herein by reference.

In order that the invention may be more fully understood three different embodiments thereof will now be described with reference to the accompanying drawings.

### BRIEF DESCRIPTION OF THE DRAWINGS

In the drawings:—

FIG. 1 is a perspective view from the top and from one side of one embodiment of scalpel having a manually operable slider according to the invention and a communicating slot extending along the top edge of the scalpel;

FIG. 2 illustrates in similar view the exposed part only of the manually operable slider for purposes of clarity;

FIG. 3 is an enlarged cross-section of this embodiment of the invention taken along line III to III in FIG. 1;

FIG. 4 is a view similar to FIG. 2 but illustrating a second embodiment of the invention being a variation of the first embodiment in which the flanges of the channel shaped slider are substantially longer;

FIG. 5 is a perspective view from the side of a third embodiment of the invention in which the communicating slot extends along the one side of the scalpel; and,

FIG. 6 is an enlarged cross-section of the third embodiment of the invention taken along line VI to VI FIG. 5.

### DETAILED DESCRIPTION WITH REFERENCE TO THE DRAWINGS

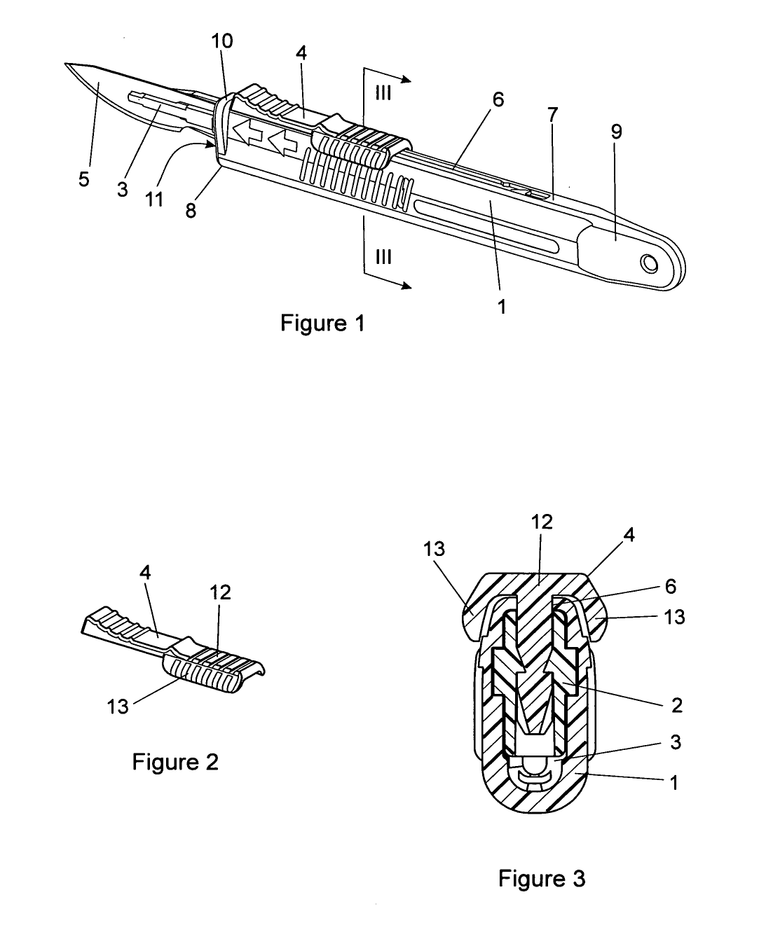

In the embodiment of the invention illustrated in FIGS. 1 to 3 of the drawings, a scalpel comprises a single piece injection moulded plastics handle (1); an injection moulded blade carrier (2) slidable longitudinally in a longitudinal cavity (3) within the plastics handle; a separately injection moulded, manually operable slider (4) that combines with the blade carrier to form a blade carrier assembly in the assembled condition; and, for use, a scalpel blade (5) that is fitted to the blade carrier.

The handle has a longitudinally extending slot (6) extending along its operatively top edge (7) from a forward end (8) of the handle towards a rear end (9) thereof and communicating with the cavity inside. An integral bridge (10) at the front end of the top edge forms an endless collar to an open end (11) to the cavity and provides dimensional stability to this end for firmly holding the blade carrier in its operative position. Apart from the slider that is further described below, the scalpel generally has features described in the published United States patent application referred to above.

As provided by this invention, the slider is of generally channel shape in cross-section with a web section (12) extending across the upper edge of the scalpel handle and a flange section (13) extending one over the adjacent part of each side of the handle. The outer surfaces of the flanges can be provided with undulations for enhancing frictional engagement thereof by a thumb or finger of a person.

In the embodiment of the invention illustrated in FIGS. 1 to 3, the flanges extend only a short distance down the side surface of the handle, as shown most clearly in FIG. 3, but I nevertheless engagable by a thumb or finger from either side of the scalpel or from the top.

It will thus be understood that this arrangement enables the slider to be operated from either side or from the top, as may be convenient to a person holding the scalpel, and depending on the orientation in which the scalpel is held.

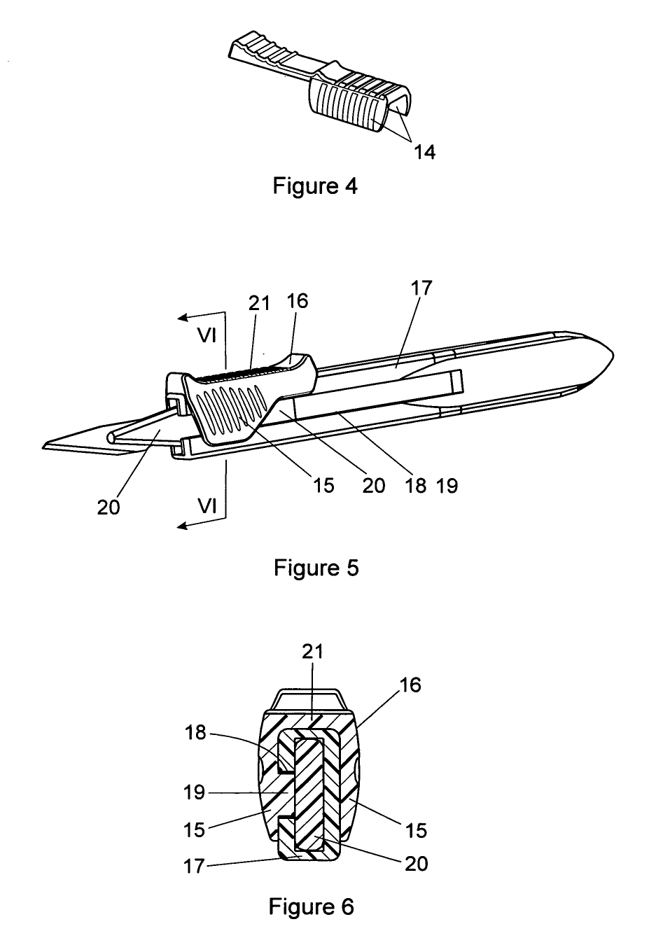

The distance by which the flanges extend over the width of the side surface of the handle is variable and, accordingly, as in the case of the second embodiment of the invention it is illustrated in FIG. 4, the flanges (14) could be substantially longer to extend over a greater part of the width of the side surface of the scalpel.

Turning now to the embodiment of the invention illustrated in FIGS. 5 and 6, the flanges (15) of the slider (16) may extend even further across the sides of the handle, and almost over the entire width of each side of the scalpel handle (17). This variation of slider is considered to be most appropriate in the instance in which a communicating slot (18) is provided in one side of the handle, typically approximately centrally along the width of the handle, so that a protrusion or other formation (19) formed integral with the slider can be attached by any suitable means to the internal blade carrier (20). The flanges (15), as in the other embodiments of the invention described above, are interconnected by a web (21) bridging one edge, herein referred to as the top edge, of the handle. Both the web and the flanges are provided with friction affording undulations, as indicated above.

It will be understood that numerous variations may be made to the embodiments of the invention described above without departing from the scope hereof. In particular, the invention can be applied to any suitable configuration of scalpel and is not limited in application to any particular scalpel.

1. A scalpel having a handle with a longitudinally extending cavity therein,
a blade carrier within the cavity and movable longitudinally relative to the handle between an operative position in which a blade carried thereby is exposed for use at an open end of the cavity and an inoperative position in which a blade carried thereby is retracted within the cavity in the handle,
and a manually operable slider associated with the blade carrier and passing through a slot in a wall of the handle,
wherein the slider is of generally channel shape in cross-section so as to have a web section and a flange section on each side thereof,
and wherein the web section bridges an edge of the handle and the flange sections are located one on each side of the edge of the handle such that the slider is operable, by the operative engagement of a thumb or finger therewith, from the top of the scalpel handle as well as from substantially either side of the scalpel handle.

2. A scalpel as claimed in claim 1 in which the slot extends along said edge of the handle.

3. A scalpel as claimed in claim 1 in which the slot extends along one side of the handle.

4. A scalpel as claimed in claim 1 in which the slider and one or other longitudinally extending edge of the slot is provided with co-operating tooth and notch formations that cooperate to releasably hold the blade carrier and slider assembly in “click-stop” manner in the operative and inoperative positions.
